(19) United States
(12) Patent Application Publication
(10) Pub. No.: US 2005/0175569 A1
(43) Pub. Date: Aug. 11, 2005

(54) Cosmetic compositions comprising a cation, a drawing polymer and a thickener, and cosmetic treatment processes

(76) Inventor: Geraldine Fack, Levallois (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/030,171

(22) Filed: Jan. 7, 2005

Related U.S. Application Data
(60) Provisional application No. 60/537,554, filed on Jan. 21, 2004.
(60) Provisional application No. 60/538,249, filed on Jan. 23, 2004.

(30) Foreign Application Priority Data
Jan. 7, 2004 (FR) 0450028
Jan. 7, 2004 (FR) 0450034

(51) Int. Cl. A61K 7/06

(52) U.S. Cl. 424/70.13

(57) ABSTRACT

The present disclosure relates to cosmetic compositions comprising, in a cosmetically acceptable medium, at least one cationic surfactant, at least one drawing polymer, and at least one non-cellulose-based thickening polymer, and to cosmetic processes for treating keratin materials, such as the hair. These compositions have an improved styling effect.

This application claims benefit of U.S. Provisional Application No. 60/537,554, filed Jan. 21, 2004, and U.S. Provisional Application No. 60/538,249, filed Jan. 23, 2004.

The present disclosure relates to cosmetic compositions, for example for conditioning the hair, comprising at least one cationic surfactant, at least one non-cellulose-based thickening polymer, and at least one drawing polymer, and to cosmetic processes for treating keratin materials, such as the hair.

It is well known that hair that is damaged, brittle, or sensitized to varying degrees under the action of atmospheric agents or under the action of mechanical or chemical treatments, such as dyeing, bleaching and/or permanent-waving, is often difficult to disentangle and to style, and lacks softness.

Cosmetic compositions comprising cationic surfactants and thickening polysaccharides, such as starch and celluloses, have already been proposed for treating keratin materials, for example the hair.

However, such compositions may have drawbacks. For example, they may be difficult to rinse out, unstable, and difficult to distribute evenly over the keratin materials, and they may have insufficient cosmetic properties.

The use of cationic polymers, cationic silicones or cationic surfactants in compositions for washing or caring for keratin materials, such as the hair, to facilitate the disentangling of the hair and to give it softness and suppleness, has previously been recommended. However, the use of cationic polymers or cations for this purpose has various drawbacks. On account of their high affinity for the hair, some of these polymers become deposited in substantial amount during repeated use, and lead to undesirable effects such as an unpleasant, laden feel, stiffening of the hair, and adhesion between the fibers that affects styling.

Furthermore, it is usually necessary to use a second product to give the hair a shape and to fix the hairstyle.

In summary, the current conditioning cosmetic compositions are not always satisfactory. Thus, the applicant sought to obtain cosmetic compositions with rinse-out styling properties equivalent to those of fixing gels or lacquers, while at the same time having good conditioning properties.

The applicant has discovered that combining a cationic surfactant, certain polymers with a particular drawing power, and a non-cellulose-based thickening polymer may make it possible to overcome these drawbacks. These compositions may be included, for example, in non-detergent media with a low or zero concentration of washing surfactants.

Hair treated with compositions according to the disclosure may be at least one of the following: smooth, easily disentangled, shiny, supple, having separate strands, soft to the touch, and without any residue. The hair may have a natural, unladen appearance.

Furthermore, the compositions are easy to rinse out.

Thus, according to the present disclosure, in one embodiment, novel non-washing cosmetic compositions are now proposed, comprising, in a cosmetically acceptable aqueous medium, at least one cationic surfactant, at least one non-cellulose-based thickening polymer, and at least one polymer with a drawing power of greater than 5 cm.

Another subject of the disclosure are cosmetic processes for treating keratin materials, such as the hair, using the compositions described above.

A subject of the disclosure is also the use of the said compositions as a conditioner.

Other subjects, characteristics, aspects, and advantages of the disclosure will emerge even more clearly on reading the description and the various examples that follow.

The term “drawing power of a polymer,” as used herein, means the length of polymer yarn obtained at the breaking point of the said yarn according to the procedure defined below.

The drawing power of the polymers that may be used according to the disclosure is the power measured for a composition containing, as a percent by weight:

| | |
|---|---|
| Sodium lauryl ether sulfate oxyethylenated | 12.5% |
| with 2.2 mol of ethylene oxide | |
| Cocoamidopropylbetaine | 2.5% |
| Test polymer | 1% |
| Water qs | 100% |

The drawing power is measured using a TA-TX2 texture analyser (Rheo/stable Micro Systems).

The measurement is performed after compression of the product:
displacement of the disc (35 mm aluminium cylinder) at a speed of 2.5 mm/s and detection of the compression strength
penetration into the product at the same speed to a depth of 10 mm
removal of the probe at a speed of 2.5 mm/s
measurement of the displacement of the probe and detection of the breaking point of the product yarn.

According to the present disclosure, the polymers with a drawing power of greater than 5 cm will be referred to as “drawing polymers.”

The at least one polymer with a drawing power of greater than 5 cm may be chosen from (a1) a dispersion of particles of a water-soluble cationic polymer with a weight-average molecular mass of greater than 10⁶ in a saline aqueous solution, obtained by heterogeneous free-radical polymerization of water-soluble monomers with precipitation of the polymer formed, and (a2) an aqueous solution of a water-soluble cationic polymer with a weight-average molecular mass of greater than 10⁶, obtained by heterogeneous free-radical polymerization of water-soluble monomers with precipitation of the polymer formed.

The term “cationic polymer,” as used herein, includes polymers comprising cationic monomers and optionally nonionic monomers.

High molecular weight water-soluble cationic polymers are polyelectrolytes polymerized using at least one cationic monomer of formula (I).

The synthesis of the high molecular weight water-soluble polymers, used in the present disclosure, takes place by heterogeneous free-radical polymerization of water-soluble monomers comprising at least one ethylenic unsaturation. The polymerization takes place in an aqueous solution of a mineral electrolyte, such as a salt, having an ionic strength that is sufficient to cause precipitation of the polymer formed as soon as it has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which may be greater than 10⁶.

The technique of heterogeneous free-radical polymerization in aqueous medium with precipitation of the polymer formed is described, for example, in U.S. Pat. No. 4,929,655, in European Patent Application No. EP 0,943,628, and in document WO 02/34796.

To ensure the stability of the polymer particle dispersions during synthesis and storage, it may be useful to perform the polymerization in the presence of a dispersant. This dispersant may be, for example, a polyelectrolyte, which, unlike the high molecular weight polymer used in the disclosure, is soluble in aqueous polymerization media of high ionic strength.

This dispersing polyelectrolyte may have, for instance, a charge identical to that of the polymer synthesized. In other words, for the synthesis of cationic polyelectrolytes, a cationic dispersing polyelectrolyte may be used.

Dispersants that may be used according to the disclosure include, but are not limited to, the cationic polyelectrolytes obtained by polymerization of 50 to 100 mole percent of at least one cationic monomer chosen from salts, for example hydrochlorides and sulfates, of dimethylaminoethyl (meth)acrylate, N-dimethylaminopropyl(meth)-acrylamide, and di(meth)allylamine; (meth)acryloyloxy-ethyltrimethylammonium chloride; (meth)acrylamido-propyltrimethylammonium chloride; and dimethyidiallyl-ammonium chloride; and of 50 to 0 mole percent of acrylamide. A polyamine such as a polyalkyleneamine may also be used.

The dispersant may be used, for example, in a proportion ranging from 1% to 10% by weight, relative to the total weight of the monomers to be polymerized.

The saline aqueous solution that serves as a synthesis and dispersion medium for the high molecular weight water-soluble polymer may be a solution of at least one mineral salt, chosen, for example, from divalent anionic salts. Examples of anionic salts that may be mentioned include, but are not limited to, ammonium sulfate, ammonium hydrogen sulfate, sodium sulfate, sodium hydrogen sulfate, magnesium sulfate, magnesium hydrogen sulfate, aluminium sulfate, and aluminium hydrogen sulfate. In one embodiment of the disclosure, the at least one mineral salt is chosen from ammonium sulfate and sodium sulfate.

The concentration of the at least one salt should be sufficient to induce the precipitation of the water-soluble polymer formed in the polymerization medium, and may be up to the saturation concentration of each salt. To obtain such a precipitation, the salt concentration may be, for example, at least equal to 10% by weight, such as greater than 15% by weight, and may be less than 50% by weight, relative to the total weight of the polymer solution or dispersion. The saline aqueous solution may also comprise monovalent salts such as sodium chloride and ammonium chloride.

The heterogeneous free-radical polymerization in aqueous medium as described above may be accompanied by a large increase in the viscosity of the reaction medium, which is reflected by difficulties in stirring, a lack of homogeneity of the reaction medium, and an increase in the particle size of the polymer particles formed. To prevent such an increase in viscosity, it has been proposed, in European Patent Application No. EP 0,943,628, that the polymerization medium comprise at least one agent to prevent the increase in viscosity of the reaction medium during polymerization.

The high molecular weight water-soluble polymers used in the present disclosure may be prepared, for example, in the presence of an agent that can prevent an increase in viscosity.

As a non-limiting example, agents that can prevent an increase in viscosity of the reaction medium may be chosen from:

(A) polycarboxylic acids and salts thereof;

(B) polyphenols;

(C) cyclic compounds comprising a hydroxyl group and a carboxyl group, and salts thereof;

(D) gluconic acid and salts thereof;

(E) the reaction products obtained by reacting a methoxyhydroquinone and/or a cationic (meth)acrylic monomer with a free-radical-generating compound, under an oxidizing atmosphere;

(F) the reaction products obtained by reacting a cationic (meth)acrylic polymer with a free-radical-generating compound, under an oxidizing atmosphere;

(G) the reaction products obtained by reacting a cationic (meth)acrylic polymer with an oxidizing agent; and mixtures thereof.

The addition of at least one agent that can prevent the increase in viscosity as described above makes it possible to perform the polymerization of the water-soluble monomers described above with a low-power stirrer while at the same time avoiding the formation of coarse particles. The agents that can prevent an increase in viscosity may be, for example, soluble in the aqueous reaction medium.

Examples of compounds (A) that may be mentioned include, but are not limited to, oxalic acid, adipic acid, tartaric acid, malic acid, phthalic acid, and the salts thereof.

Examples of compounds (B) that may be mentioned include, but are not limited to, resorcinol and pyrogallol.

Examples of compounds (C) that may be mentioned include, but are not limited to, m-hydroxybenzoic acid, p-hydroxy-benzoic acid, salicylic acid, gallic acid, tannic acid, and the salts of these acids.

Examples of compounds (D) that may be mentioned include, but are not limited to, sodium gluconate, potassium gluconate, ammonium gluconate, and various amine salts of gluconic acid.

Examples of compounds (E) that may be mentioned include, but are not limited to, those obtained by reacting a free-radical-generating compound, under a stream of oxygenated gas, in a solution comprising methoxy-hydroquinone and/or a cationic (meth)acrylic monomer. The free-radical-generating compound may be an initiator commonly used for free-radical polymerization. Examples of free-radical-generating compounds that may be mentioned include, but are not limited to, water-soluble azo initiators such as 2,2′-azobis(2-amidinopropane) hydrochloride sold, for example, under the name V-50 by the company Wako Chemical Industries, and 2,2′-azobis[2-(2-imidazolin-2-yl)propane] hydrochloride sold, for example, under the trade name VA-044 by the company Wako Chemical Industries; and initiators from the group of water-soluble redox agents, such as the ammonium persulfate/sodium hydrogen sulfite combination.

Agents that can prevent an increase in viscosity (F) may be obtained by reacting a free-radical initiator, under an oxygenated atmosphere, with a dispersant according to the disclosure. The polymerization initiator may be chosen from water-soluble azo initiators and water-soluble redox agents as described above.

The compounds (G) may be obtained in the form of oxidized polymers of low molecular mass by oxidation of a cationic dispersant according to the disclosure obtained by polymerization of a cationic (meth)acrylic monomer, using an oxidizing agent such as hydrogen peroxide or a halogen.

As cationic (meth)acrylic monomers used for the preparation of agents that can prevent an increase in viscosity (E), (F) and (G), examples that may be mentioned include, but are not limited to, dimethylaminoethyl (meth)acrylate hydrochloride; dimethylaminoethyl (meth)acrylate sulfate; (meth)acryloyloxyethyl-trimethylammonium chloride; (meth)acryloyloxyethyl-dimethylbenzylammonium chloride; the hydrochloride and sulfate derived from N-dimethylaminopropyl(meth)-acrylamide; (meth)acrylamidopropyltrimethylammonium chloride, dimethylaminohydroxypropyl (meth)acrylate chloride; dimethylaminohyd roxypropyl (meth )acrylate sulfate; (meth)acryloyloxyhydroxypropyl-trimethylammoniu m chloride; and (meth)acryloyloxy-hydroxypropyldimethylbenzylammonium chloride.

Agents (A) to (G), which prevent an increase in viscosity, may be used alone or as mixtures, in an amount ranging, for example, from 10 ppm to 10,000 ppm, relative to the total weight of the reaction solution.

The water-soluble monomers polymerized by heterogeneous free-radical polymerization to obtain high molecular weight water-soluble polymers may be monomers comprising at least one ethylenic double bond, for example a vinyl, acrylic or allylic double bond. They may be cationic or non-ionic, and may be used as a mixture.

Water-soluble nonionic monomers that may be mentioned include acrylamide, methacrylamide, N-vinylformamide, N-vinylacetonamide, hydroxypropyl acrylate, and hydroxypropyl methacrylate.

The water-soluble cationic monomers may be chosen, for example, from di(C₁₋₄ alkyl)diallylammonium salts and the compounds of formula (I) below: 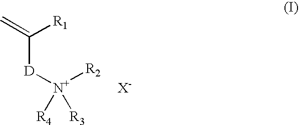
wherein:
R₁ is chosen from hydrogen and methyl;
R₂ and R₃, which may be identical or different, are chosen from hydrogen and linear and branched C₁₋₄ alkyl groups;
R₄ is chosen from hydrogen, linear and branched C₁₋₄ alkyl groups, and aryl groups; and
D is chosen from units of the formula: 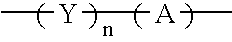
   wherein:
   Y is chosen from amide (—CO—NH—), ester (—O—CO— or —CO—O—), urethane (—O—CO—NH—), and urea (—NH—CO—NH—) functions;
   A is chosen from linear, branched, and cyclic C₁₋₁₀ alkylene groups, which may be substituted or interrupted with a divalent aromatic or heteroaromatic ring, or which may be interrupted with a heteroatom chosen from O, N, S and P, and which may comprise a ketone, amide, ester, urethane or urea function;
   n is 0 or 1; and
X⁻ is chosen from anionic counterions, such as halide, for example chloride, and sulfate ions.

Examples of water-soluble cationic monomers that may be mentioned include, but are not limited to, dimethylaminoethyl (meth)acrylate hydrochloride, dimethylaminoethyl (meth)acrylate sulfate, (meth)acryloyloxyethyltrimethylammoniu m chloride, (meth)acryloyloxyethyidimethylbenzylammonium chloride, N-dimethylaminopropyl(meth)acrylamide hydrochloride, N-dimethylaminopropyl(meth)acrylamide sulfate, (meth)acrylamidopropyltrimethylammonium chloride, (meth)acrylamidopropyidimethylbenzylammonium chloride, dimethylaminohydroxypropyl (meth)acrylate hydrochloride, dimethylaminohydroxypropyl (meth)acrylate sulfate, (meth)acryloyloxyhydroxy-propyltrimethylammonium chloride, (meth)acryloyloxy-hydroxypropyldimethylbenzylammonium chloride, and dimethyldiallylammonium chloride.

In one embodiment of the present disclosure, the high molecular weight water-soluble polymer is obtained by heterogeneous free-radical polymerization of a monomer mixture comprising from 0 to 95.5 mole percent of acrylamide and from 4.5 to 100 mole percent of at least one cationic monomer of formula (I).

According to another embodiment, the water-soluble polymers may be obtained by polymerization of a monomer mixture comprising acrylic acid and a cationic monomer of formula (I), in which the number of moles of cationic monomer of formula (I) is greater than the number of moles of acrylamide.

Water-soluble polyelectrolytes that may be used according to the disclosure include, as non-limiting examples, those polymerized using monomer mixtures consisting, respectively, of:
1. 10 mole percent of acryloyloxyethyldimethylbenzyl-ammonium chloride and 90 mole percent of acrylamide;
2. 30 mole percent of acryloyloxytrimethylammonium chloride, 50 mole percent of acryloyloxyethyldimethylbenzyl-ammonium chloride, and 20 mole percent of acrylamide;
3. 10 mole percent of acryloyloxyethyltrimethylammonium chloride and 90 mole percent of acrylamide;
4. 30 mole percent of diallyldimethylammonium chloride and 70 mole percent of acrylamide.

The water-soluble polymers used in the present disclosure have a weight-average molecular mass of greater than 1,000,000, ranging, for example, from 1,000,000 to 50,000,000. This weight-average molecular mass may be determined via the RSV (Reduced Specific Viscosity) method as defined in “Principles of Polymer Chemistry,” Cornell University Press, Ithaca, NY, 1953, chapter VII, entitled “Determination of Molecular Weight,” pages 266-316.

The amount of the high molecular weight water-soluble cationic polymer dispersion or solution may be chosen, for example, such that the at least one water-soluble cationic polymer is present in an amount ranging from 0.01% to 10% by weight, relative to the total weight of the final composition, for instance from 0.05% to 5% by weight, relative to the total weight of the final composition.

The concentration of particles of the at least one water-soluble cationic polymer present as a dispersion in a saline aqueous solution in (al) may range from 0.01% to 20% by weight, relative to the total weight of the dispersion.

The concentration of the at least one water-soluble cationic polymer in a saline aqueous solution in (a2) ranges from 0.01% to 20% by weight, relative to the total weight of the solution.

The at least one drawing polymer according to the disclosure can be present in the composition in an amount ranging, for example, from 0.01% to 10% by weight, relative to the total weight of the final composition, such as from 0.05% to 5% by weight, relative to the total weight of the final composition.

The compositions according to the disclosure comprise at least one cationic surfactant that is well known per se, such as optionally polyoxyalkylenated primary, secondary or tertiary fatty amine salts, quaternary ammonium salts, and mixtures thereof.

Examples of quaternary ammonium salts that may be mentioned include, but are not limited to:
those of general formula (V) below: 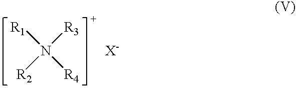
   wherein
   R₁, R₂, R₃, and R₄, which may be identical or different, are chosen from linear and branched aliphatic radicals comprising from 1 to 30 carbon atoms and optionally comprising heteroatoms such as oxygen, nitrogen, sulfur and halogens. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals; and aromatic radicals such as aryl and alkylaryl;
X⁻ is chosen from halides, phosphates, acetates, lactates, (C₂-C₆) alkyl sulfates and alkyl- or alkylaryl-sulfonates;
   quaternary ammonium salts of imidazoline, for instance those of formula (VI) below: 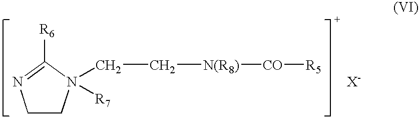
      wherein:
      R₅ is chosen from alkenyl and alkyl radicals comprising from 8 to 30 carbon atoms, for example fatty acid derivatives of tallow or of coconut;
      R₆ is chosen from hydrogen, C₁-C₄ alkyl radicals, and alkenyl and alkyl radicals comprising from 8 to 30 carbon atoms;
      R₇ is chosen from C₁-C₄ alkyl radicals;
      R₈ is chosen from hydrogen and C₁-C₄ alkyl radicals; and
      X⁻¹ is chosen from halides, phosphates, acetates, lactates, alkyl sulfates, alkyl sulfonates, and alkylaryl sulfonates.
         In one embodiment, R₅ and R₆ are, for example, a mixture of radicals chosen from alkenyl and alkyl radicals comprising from 12 to 21 carbon atoms, such as fatty acid derivatives of tallow, R₇ is methyl and R₈ is hydrogen. Examples of such products include, but are not limited to, Quaternium-27 (CTFA 1997) and Quaternium-83 (CTFA 1997), which are sold under the names “Rewoquat®” W75, W90, W75PG and W75HPG by the company Witco.
diquaternary ammonium salts of formula (VII): 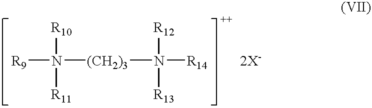
   wherein:
   ₉ is chosen from aliphatic radicals comprising from 16 to 30 carbon atoms;
   R₁₀, R₁₁, R₁₂, R₁₃, and R₁₄, which may be identical or different, are chosen from hydrogen and alkyl radicals comprising from 1 to 4 carbon atoms; and
   X⁻ is chosen from halides, acetates, phosphates, nitrates, ethyl sulfates, and methyl sulfates.
      An example of one such diquaternary ammonium salt is propanetallowdiammonium dichloride.
quaternary ammonium salts comprising at least one ester function, such as those of formula (VIII) below: 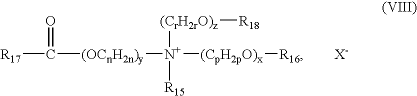
   wherein:
   R₁₅ is chosen from C₁-C₆ alkyl radicals and C₁-C₆ hydroxyalkyl and dihydroxyalkyl radicals;
   R₁₆ is chosen from:
      a radical 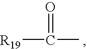
      linear and branched, saturated and unsaturated C₁-C₂₂ hydrocarbon-based radicals R₂₀, and
      hydrogen,
   R₁₈ is chosen from:
      a radical 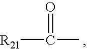
      linear and branched, saturated and unsaturated C₁-C₆ hydrocarbon-based radicals R₂₂, and
      hydrogen,
   R₁₇, R₁₉, and R₂₁, which may be identical or different, are chosen from linear and branched, saturated and unsaturated, C₇-C₂₁, hydrocarbon-based radicals;
   r, n, and p, which may be identical or different, are chosen from integers ranging from 2 to 6;
   y is chosen from integers ranging from 1 to 10;
   x and z, which may be identical or different, are chosen from integers ranging from 0 to 10;
   X⁻ is chosen from simple and complex, organic and inorganic anions; with the proviso that the sum x+y+z ranges from 1 to 15, that when x is 0, R₁₆ denotes R₂₀, and that when z is 0, R₁₈ denotes R₂₂.

R₁₅ may be chosen from linear and branched alkyl radicals. In one embodiment, R₁₅ is chosen from linear alkyl radicals.

In another embodiment, R₁₅ is chosen from methyl, ethyl, hydroxyethyl, and dihydroxypropyl radicals, for example methyl and ethyl radicals.

In one embodiment, the sum x+y+z ranges from 1 to 10.

When R₁₆ is a hydrocarbon-based radical R₂₀, it may be long and comprise from 12 to 22 carbon atoms, or short and comprise from 1 to 3 carbon atoms.

When R₁₈ is a hydrocarbon-based radical R₂₂, it may comprise, for example, from 1 to 3 carbon atoms.

By way of non-limiting example, in one embodiment, R₁₇, R₁₉, and R₂₁, which may be identical or different, are chosen from linear and branched, saturated and unsaturated, C₁₁-C₂₁ hydrocarbon-based radicals, for example from linear and branched, saturated and unsaturated C₁₁-C₂₁ alkyl and alkenyl radicals.

In another embodiment, x and z, which may be identical or different, are 0 or 1.

In one embodiment, y is equal to 1.

In another embodiment, r, n and p, which may be identical or different, are equal to 2 or 3, for example equal to 2.

The anion X⁻ may be chosen from, for example, halides, such as chloride, bromide, and iodide; and C1-C4 alkyl sulfates, such as methyl sulfate. However, methanesulfonate, phosphate, nitrate, tosylate, an anion derived from an organic acid, such as acetate and lactate, and any other anion that is compatible with the ammonium comprising an ester function, are other non-limiting examples of anions that may be used according to the disclosure.

In one embodiment, the anion X⁻ is chosen from chloride and methyl sulfate.

In another embodiment, the ammonium salts of formula (VIII) may be used, wherein:
R₁₅ is chosen from methyl and ethyl radicals,
x and y are equal to 1;
z is equal to 0 or 1;
r, n and p are equal to 2;
R₁₆ is chosen from:
   a radical 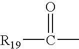
   methyl, ethyl, and C₁₄-C₂₂ hydrocarbon-based radicals,
   hydrogen;
R₁₈ is chosen from:
   a radical 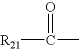
   hydrogen;
R₁₇, R₁₉, and R₂₁, which may be identical or different, are chosen from linear and branched, saturated and unsaturated, C₁₃-C₁₇ hydrocarbon-based radicals, for example from linear and branched, saturated and unsaturated, C₁₃-C₁₇ alkyl and alkenyl radicals.

In one embodiment, the hydrocarbon-based radicals are linear.

Non-limiting examples of compounds of formula (VII) that may be mentioned include salts, for example chloride and methyl sulfate, of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. In one embodiment, the acyl radicals may comprise from 14 to 18 carbon atoms, and may be derived, for example, from a plant oil, for instance palm oil and sunflower oil. When the compound comprises several acyl radicals, these radicals may be identical or different.

These products may be obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification may be followed by a quaternization using an alkylating agent chosen from alkyl halides, for example methyl and ethyl halides; dialkyl sulfates, for example dimethyl and diethyl sulfates; methyl methanesulfonate; methyl para-toluenesulfonate; glycol chlorohydrin; and glycerol chlorohydrin.

Such compounds are sold, for example, under the names Dehyquart® by the company Cognis, Stepanquat® by the company Stepan, Noxamium® by the company Ceca, and “Rewoquat® WE 18” by the company Rewo-Goldschmidt.

The compositions according to the disclosure may comprise, for example, a mixture of quaternary ammonium mono-, di- and triester salts with a weight majority of diester salts.

A non-limiting example of a mixture of ammonium salts that may be used in the compositions according to the disclosure is that comprising from 15% to 30% by weight of acyloxyethyl-dihydroxyethyl-methylammonium methyl sulfate, from 45% to 60% by weight of diacyloxyethyl-hydroxyethyl-methylammonium methyl sulfate, and from 15% to 30% by weight of triacyloxyethyl-methylammonium methyl sulfate, the acyl radicals of all these compounds comprising from 14 to 18 carbon atoms and being derived from optionally partially hydrogenated palm oil.

Other non-limiting examples of ammonium salts that may be used in the compositions according to the disclosure include the ammonium salts comprising at least one ester function described in U.S. Pat. Nos. 4,874,554 and 4,137,180.

Among the quaternary ammonium salts mentioned above that may be used in compositions according to the disclosure include, but are not limited to, those corresponding to formula (V), for example tetraalkylammonium chlorides, for instance dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the at least one cationic surfactant that may be used in the compositions of the disclosure are chosen from quaternary ammonium salts, for example from behenyltrimethylammonium chloride, cetyltrimethylammonium chloride, Quaternium-83, behenylamidopropyl-2,3-dihydroxypropyldimethylammonium chloride, and palmitylamidopropyltrimethylammonium chloride.

The compositions according to the disclosure may comprise the at least one cationic surfactant in an amount ranging from 0.05% to 10% by weight, relative to the total weight of the composition, for example from 0.1% to 8% by weight, relative to the total weight of the composition, such as from 0.2% to 5% by weight, relative to the total weight of the composition.

According to the disclosure, the term “non-cellulose-based thickening polymer” means a thickening polymer not comprising any cellulose units.

The at least one non-cellulose-based thickening polymer is different from the high molecular weight water-soluble cationic polymer according to the disclosure.

The at least one non-cellulose-based thickener according to the disclosure may be of natural or synthetic origin. It may be chosen, for example, from:

(i) homopolymers and copolymers comprising ethylenically unsaturated monomers of ester and/or amide type,

(ii) vinylpyrrolidone homopolymers and copolymers, and

(iii) non-cellulose-based polysaccharides.

The purely synthetic thickeners according to the disclosure may be chosen, for example, from acrylic and/or methacrylic acid polymers and copolymers, for instance acrylic acid/ethyl acrylate copolymers and carboxyvinyl polymers. Non-limiting examples of such polymers and copolymers include the “carbomer” products (CTFA) sold by the company Goodrich under the names Carbopol (Carbopol 980, 981, 954, 2984 and 5984) and Synthalen, the polyglyceryl methacrylate sold by the company Guardian under the name Lubragel, and the polyglyceryl acrylate sold under the name Hispagel by the company Hispano Chimica.

Polyethylene glycols (PEG) and derivatives thereof may also be used as thickeners.

According to one embodiment, the following may be used as thickeners:
crosslinked 2-acrylamido-2-methylpropane sulfonic homopolymers and copolymers,
optionally crosslinked copolymers of acrylamide and of ammonium acrylate,
optionally crosslinked homopolymers and copolymers of methacryloyl-oxyethyltrimethylammonium chloride, and
optionally crosslinked, partially or totally neutralized crosslinked copolymers of acrylamide and of 2-acrylamido-2-methylpropanesulfonic acid.

Among the crosslinked acrylamide/ammonium acrylate copolymers used in accordance with the present disclosure, non-limiting mention may be made of acrylamide/ammonium acrylate (5/95 by weight) copolymers crosslinked with a crosslinking agent comprising olefinic polyunsaturation, such as divinylbenzene, tetraallyloxyethane, methylenebisacrylamide, diallyl ether, polyallylpolyglyceryl ethers, and allylic ethers of alcohols of the sugar series, such as erythritol, pentaerythritol, arabitol, mannitol, sorbitol and glucose.

Non-limiting examples of similar copolymers are described and prepared in French Patent No. 2,416,723 and U.S. Pat. Nos. 2,798,053 and 2,923,692.

This type of crosslinked copolymer may be used, for example, in the form of a water-in-oil emulsion comprising about 30% by weight of the said copolymer, 25% by weight of liquid paraffin, 4% by weight of a mixture of sorbitan stearate and of a hydrophilic ethoxylated derivative, and 41% by weight of water. Such an emulsion is sold under the name “Bozepol C” by the company Hoechst.

The copolymers of acrylamide and of 2-acrylamido-2-methylpropanesulfonic acid used in accordance with the present disclosure may be copolymers crosslinked with a compound comprising olefinic polyunsaturation, such as those mentioned above, and partially or totally neutralized with a neutralizer chosen, for example, from sodium hydroxide, potassium hydroxide, aqueous ammonia, and amines such as triethanolamine and monoethanolamine.

They may be prepared by copolymerizing acrylamide and sodium 2-acrylamido-2-methylpropane sulfonate, via a free radical route, using initiators such as azobisisobutyronitrile and by precipitation from an alcohol such as tert-butanol.

As non-limiting examples, copolymers obtained by copolymerization of from 70 mole percent to 55 mole percent of acrylamide and from 30 mole percent to 45 mole percent of sodium 2-acrylamido-2-methylpropane sulfonate may be used. The crosslinking agent may be used at concentrations ranging from 10⁻⁴ to 4×10⁻⁴ mol per mole of the monomer mixture.

In one embodiment, these particular copolymers may be incorporated into the compositions of the disclosure in the form of water-in-oil emulsions comprising from 35% to 40% by weight of this copolymer, from 15% to 25% by weight of a mixture of C₁₂-C₁₃ isoparaffinic hydrocarbons, from 3% to 8% by weight of polyethylene glycol lauryl ether comprising 7 mol of ethylene oxide, and water. Such an emulsion is sold, for example, under the name “Sepigel 305” by the company SEPPIC.

A non-limiting example of the crosslinked copolymer of acrylamide and of methacryloyloxyethyltrimethylammonium chloride used according to the disclosure is a copolymer obtained by copolymerization of acrylamide and of dimethylaminoethyl methacrylate quaternized with methyl chloride, followed by crosslinking with an olefinically unsaturated compound, for example methylenebisacrylamide.

In one embodiment, crosslinked acrylamide/methacryloyloxy-ethyltrimethylammonium chloride copolymers, comprising about 50% by weight of each polymer, can be used in the form of dispersions comprising 50% by weight of the said copolymer in mineral oil. One such dispersion is sold under the name “Salcare SC92” by the company Ciba.

In another embodiment, crosslinked methacryloyloxyethyltrimethyl-ammonium chloride homopolymers, for example in the form of inverse dispersions, may be used. These dispersions are sold under the name “Salcare SC95” and “Salcare SC96” by the company Ciba.

Non-limiting examples of non-crosslinked copolymers of methacrylamide and of methacryloyloxyethyltrimethylammonium chloride include the products sold under the trade names “Rohagit KF 400” and “KF 720” by the company Rohm & Haas.

The homopolymers or copolymers comprising ethylenically unsaturated monomers of ester and/or amide type that may be mentioned include, but are not limited to, polyamides, for example polyacrylamide, such as that sold under the name “Cyanamer P250” by the company Cytec (polyacrylamide); methyl methacrylate/ethylene glycol dimethacrylate copolymers, such as that sold under the name “PMMA MBX-8C” by the company US Cosmetics; butyl methacrylate/methyl methacrylate copolymers, such as that sold under the name Acryloid B66 by the company Rohm & Haas; and polymethyl methacrylate, such as that sold under the name BPA 500 by the company Kobo.

The vinylpyrrolidone homopolymers or copolymers may be chosen, for example, from crosslinked vinylpyrrolidone homopolymers such as “Polymer ACP-10” sold by ISP.

The thickening polysaccharides may be chosen, for example, from glucans; modified and unmodified starches, such as those derived, for example, from cereals, for instance wheat, corn and rice, from vegetables, for instance yellow pea, and from tubers, for instance potato and cassava; amylose; amylopectin; glycogen; dextrans; mannans; xylans; arabans; galactans; galacturonans; chitin; chitosans; glucuronoxylans; arabinoxylans; xyloglucans; glucomannans; pectic acids and pectins; arabinogalactans; carrageenans; agars; gum arabics; gum tragacanths; ghatti gums; karaya gums; carob gums; galactomannans, such as guar gums and nonionic derivatives thereof, for example hydroxypropyl guar; and mixtures thereof.

Compounds of this type that may be used in the present disclosure are chosen from those described, for example, in “Encyclopedia of Chemical Technology,” Kirk-Othmer, Third Edition, 1982, volume 3, pp. 896-900, and volume 15, pp. 439-458, in “Polymers in Nature,” by E. A. MacGregor and C. T. Greenwood, published by John Wiley & Sons, Chapter 6, pp. 240-328, 1980, and in “Industrial Gums—Polysaccharides and their Derivatives,” edited by Roy L. Whistler, Second Edition, published by Academic Press Inc. The content of these three publications is hereby entirely incorporated by reference in the present patent application.

In one embodiment, starches, guar gums and derivatives thereof are used.

The thickening polysaccharides may be modified or unmodified.

Non-limiting examples of unmodified guar gums include the products sold under the name Vidogum GH 175 by the company Unipectine and under the names Meypro-Guar 50 and Jaguar C by the company Rhodia Chimie.

In one embodiment, the thickening polysaccharides may be modified nonionic guar gums, for example those modified with C₁-C₆ hydroxyalkyl groups. The hydroxyalkyl groups that may be mentioned include, but are not limited to, hydroxymethyl, hydroxyethyl, hydroxypropyl, and hydroxybutyl groups. These guar gums are well known in the prior art and can be prepared, for example, by reacting the corresponding alkene oxides such as, for example, propylene oxides, with the guar gum, so as to obtain a guar gum modified with hydroxypropyl groups. The degree of hydroxyalkylation, which corresponds to the number of alkylene oxide molecules consumed by the number of free hydroxyl functions present on the guar gum, ranges, for example, from 0.4 to 1.2.

Such nonionic guar gums optionally modified with hydroxyalkyl groups are sold, for example, under the trade names Jaguar HP8, Jaguar HP60, Jaguar HP1 20, Jaguar DC 293, and Jaguar HP 105 by the company Rhodia Chimie, or under the name Galactasol 4H4FD2 by the company Aqualon.

In another embodiment, the at least one non-cellulose-based thickening polymer in the cosmetic compositions in accordance with the present disclosure has in solution or in dispersion, at an active material concentration of 1% in water, a viscosity measured using a Rheomat RM 180 rheometer, at 25° C., of greater than 0.1 cP, for example greater than 0.2 cP, at a shear rate of 200 s⁻¹.

According to the disclosure, the at least one non-cellulose-based thickening polymer may be present in an amount ranging from 0.001% to 20% by weight, relative to the total weight of the final composition, for example from 0.01% to 15% by weight, relative to the total weight of the final composition, such as from 0.1% to 10% by weight, relative to the total weight of the final composition.

The compositions according to the disclosure may optionally comprise surfactants other than cationic surfactants.

These surfactants may be present in an amount ranging from 0.1% to 10% by weight, relative to the total weight of the final composition, for example from 0.5% to 8% by weight, relative to the total weight of the final composition, such as from 1% to 5% by weight approximately, relative to the total weight of the composition.

The compositions according to the disclosure may comprise, for example, at least one surfactant chosen from nonionic surfactants.

The nonionic surfactants are also compounds that are well known per se (see, for example, in this respect “Handbook of Surfactants” by M. R. Porter, published by Blackie & Son (Glasgow and London), 1991, pp. 116-178) and, in the context of the present disclosure, their nature is not a critical feature. Thus, by way of non-limiting example, they can be chosen from polyethoxylated, polypropoxylated and polyglycerolated fatty alcohols; polyethoxylated, polypropoxylated and polyglycerolated alkylphenols; polyethoxylated, polypropoxylated and polyglycerolated α-diols; and polyethoxylated, polypropoxylated and polyglycerolated fatty acids, all of which may comprise a fatty chain comprising, for example, 8 to 18 carbon atoms, and all of which may comprise a number of ethylene oxide or propylene oxide groups ranging, for example, from 2 to 50 and a number of glycerol groups ranging, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average 1 to 5, for example 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides, and N-acylaminopropylmorpholine oxides. In one embodiment of the present disclosure, the nonionic surfactants are chosen from alkylpolyglycosides.

Nonionic surfactants may be present in the non-detergent compositions according to the disclosure in amounts ranging from 0. 1% to 10% by weight, relative to the total weight of the composition, for example from 0.5% to 8% by weight, relative to the total weight of the composition, such as from 1% to 5% by weight, relative to the total weight of the composition.

The non-washing compositions, that is, those with zero concentration of detergents, comprise, for example, less than 4% by weight of anionic surfactants, relative to the total weight of the composition, such as less than 1% by weight, relative to the total weight of the composition.

The compositions according to the disclosure may also comprise at least one conditioner chosen from silicones, cationic polymers, carboxylic esters comprising at least 12 carbon atoms, plant oils, mineral oils, synthetic oils such as poly(α-olefins), and mixtures thereof.

The silicones that may be used in accordance with the disclosure include, but are not limited to, those that are soluble and insoluble in the compositions, for example polyorganosiloxanes that are insoluble in the compositions of the disclosure. They may be in the form of oils, waxes, resins or gums. They may be used pure or as an emulsion, a dispersion, or a microemulsion.

The organopolysiloxanes are defined in greater detail in Walter Noll's “Chemistry and Technology of Silicones” (1968) Academic Press. They can be volatile or non-volatile.

When they are volatile, the silicones may be chosen, for example, from those having a boiling point ranging from 60° C. to 260° C., such as from:
(i) cyclic silicones comprising from 3 to 7 silicon atoms, for example from 4 to 5 silicon atoms. These silicones include, for example, octamethylcyclotetrasiloxane, sold for instance under the names “Volatile Silicone 7207” by Union Carbide and “Silbione 70045 V 2” by Rhodia, decamethylcyclopentasiloxane, sold for instance under the names “Volatile Silicone 7158” by Union Carbide and “Silbione 70045 V 5” by Rhodia, and mixtures thereof. Non-limiting mention may also be made of dimethylsiloxane/methylalkylsiloxane cyclocopolymers, such as “Silicone Volatile FZ 3109” sold by the company Union Carbide, having the chemical structure: 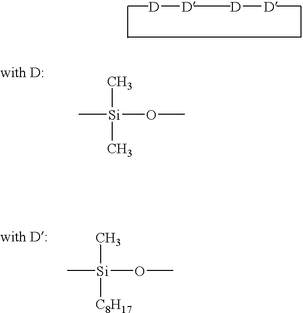
   Non-limiting mention may also be made of mixtures of cyclic silicones with organosilicon compounds, such as the 50-50 mixture of octamethylcyclotetrasiloxane and tetratrimethylsilylpentaerythritol, and the mixture of octamethylcyclotetrasiloxane and oxy-1,1′-bis(2,2,2′,2′,3,3′-hexatrimethylsilyloxy)neopentane;
(ii) linear volatile silicones comprising 2 to 9 silicon atoms and having a kinematic viscosity of less than or equal to 5×10⁻⁶ m²/s at 25° C. A non-limiting example is decamethyltetrasiloxane, sold, for example, under the name “SH 200” by the company Toray Silicone. Silicones belonging to this category are also described in the article published in Cosmetics and Toiletries, Vol. 91, Jan. 76, pp. 27-32, Todd & Byers, entitled “Volatile Silicone Fluids for Cosmetics”.

The non-volatile silicones that may be mentioned include, but are not limited to, polyalkylsiloxanes, polyarylsiloxanes, polyalkylarylsiloxanes, silicone gums and resins, polyorganosiloxanes modified with organofunctional groups, and mixtures thereof.

The organomodified silicones that can be used in accordance with the disclosure include those defined above and comprising in their structure at least one organofunctional group attached via a hydrocarbon-based group.

Among the organomodified silicones, non-limiting mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name “DC 1248,” the oils “Silwet® L 722,” “Silwet® L 7500,” “Silwet® L 77,” and “Silwet® L 711,” sold by the company Union Carbide, and the (C₁₂)alkylmethicone copolyol sold by the company Dow Corning under the name “Q2 5200”;
substituted or unsubstituted amine groups, such as the products sold under the names “GP 4 Silicone Fluid” and “GP 7100” by the company Genesee, or the products sold under the names “Q2 8220,” “Dow Corning 929,” and “Dow Corning 939” by the company Dow Corning. The substituted amine groups may be, for example, C₁-C₄ aminoalkyl groups;
thiol groups, such as the products sold under the names “GP 72 A” and “GP 71” from Genesee;
alkoxylated groups, such as the product sold under the names “Silicone Copolymer F-755” by the company SWS Silicones and “Abil Wax® 2428,” “Abil Wax® 2434,” and “Abil Wax® 2440” by the company Goldschmidt;
hydroxylated groups, such as the polyorganosiloxanes comprising a hydroxyalkyl function, described in French Patent Application No. 85/16334;
acyloxyalkyl groups, such as, for example, the polyorganosiloxanes described in U.S. Pat. No. 4,957,732;
anionic groups of carboxylic acid type, such as, for example, in the products described in European Patent No. EP 186,507 from the company Chisso Corporation, or of alkylcarboxylic type, such as those present in the product X-22-3701^{E} from the company Shin-Etsu; 2-hydroxyalkyl sulfonate; 2-hydroxyalkyl thiosulfate such as the products sold by the company Goldschmidt under the names “Abil® S201” and “Abil® S255”;
hydroxyacylamino groups, such as the polyorganosiloxanes described in European Patent Application No. EP 342,834. Non-limiting mention may be made, for example, of the product “Q2-8413” from the company Dow Corning.

In one embodiment, the silicones are chosen from polydimethylsiloxanes, polyalkylarylsiloxanes, and polydimethylsiloxanes comprising amino or alkoxylated groups.

The compositions according to the disclosure may also comprise at least one carboxylic acid ester, for instance compounds of formula RₐCOOR_{b} in which Rₐ is chosen from higher fatty acid residues comprising from 4 to 29 carbon atoms and R_{b} is chosen from hydrocarbon-based chains comprising from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyidecyl palmitate, 2-octyldodecyl myristate, 2-octyldodecyl lactate, isostearyl neopentanoate, and mixtures thereof.

The compositions according to the disclosure may also comprise at least one plant oil, such as sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, maize oil, hazelnut oil, shea butter, palm oil, apricot kernel oil, beauty-leaf oil, and mixtures thereof.

The compositions according to the disclosure may also comprise at least one mineral oil, such as liquid paraffin and liquid petroleum jelly.

The compositions according to the disclosure may comprise conditioners in an amount ranging from 0.01% to 20% by weight, relative to the total weight of the composition, for example from 0.1% to 10% by weight, relative to the total weight of the composition, such as from 0.3% to 5% by weight, relative to the total weight of the composition.

In some embodiments, the cosmetically acceptable medium may be aqueous, and may comprise water or a mixture of water and a cosmetically acceptable solvent chosen from C₁-C₄ lower alcohols, for example ethanol, isopropanol, tert-butanol, and n-butanol; polyols, for instance propylene glycol and glycerol; polyol ethers; C₅-C₁₀ alkanes; and mixtures thereof. The solvents may be chosen from, for example, glycerol and propylene glycol.

The cosmetically acceptable medium, which may be, for example, aqueous, is present in an amount ranging from 30% to 98% by weight, relative to the total weight of the composition.

The solvents may be present, for example, in amounts ranging from 0.5% to 30% by weight, relative to the total weight of the composition.

The pH of the compositions of the disclosure may range from 2 to 8, for example from 3 to 7.

The compositions according to the disclosure may also comprise standard additives that are well known in the art, such as anionic, nonionic and amphoteric polymers; non-polymeric thickeners, for instance acids and electrolytes; opacifiers; nacreous agents; vitamins, provitamins such as panthenol; waxes such as plant waxes; natural and synthetic ceramides; fragrances; dyes; organic and mineral particles; preserving agents; and pH stabilizers.

A person skilled in the art will take care to select the optional additives and the amount thereof such that they do not harm the properties of the compositions of the present disclosure.

These additives may be present in the compositions according to the disclosure in an amount ranging from 0% to 20% by weight, relative to the total weight of the composition.

The compositions of the disclosure may be in the form of rinse-out and leave-in conditioners; permanent waving, relaxing, dyeing, and bleaching compositions; or rinse-out compositions to be applied before or after a dyeing, bleaching, permanent-waving or relaxing procedure or alternatively between the two steps of a permanent-waving or relaxing procedure.

The compositions of the disclosure may be used, for example, as conditioners, care products, deep-down care masks, and scalp treatment lotions and creams. These compositions may be rinse-out and leave-in compositions.

According to one embodiment of the disclosure, the compositions may be used as conditioners, for example as conditioners on fine hair. These conditioners may be rinse-out and leave-in conditioners, for instance rinse-out conditioners.

The cosmetic compositions according to the disclosure may be in the form of a gel, a milk, a cream, an emulsion, a fluid or thickened lotion, and a foam, and may be used for the skin, the nails, the eyelashes, the lips, and the hair.

The compositions may be packaged in various forms, for example in vaporizers, pump-dispenser bottles, and in aerosol containers, in order to dispense the compositions in vaporized form or in the form of a mousse. Such packaging forms are indicated, for example, when it is desired to obtain a spray, a lacquer, or a mousse for treating the hair.

The present disclosure also relates to cosmetic processes for treating keratin materials such as, for example, the skin and the hair, comprising applying, to the keratin materials, an effective amount of a cosmetic composition comprising, in a cosmetically acceptable medium, at least one cationic surfactant, at least one non-cellulose-based thickening polymer, and at least one polymer with a drawing power of greater than 5 cm, and optionally rinsing it off after optionally leaving it to act for a period of time. Rinsing may be performed, for example, with water.

Thus, these processes according to the disclosure allow holding of the hairstyle, treatment, conditioning, and care of the hair and any other keratin material.

The disclosure may be understood more clearly with the aid of the non-limiting examples that follow, which constitute preferred embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLES 1 AND 2

The following rinse-out conditioning compositions were prepared:

| | 1 | 2 |
|---|---|---|
| Copolymer (90 mol/10 mol) of acrylamide and of | 1% AM | 0.5% AM |
| dimethylaminoethyl acrylate quaternized with | | |
| benzyl chloride (MW > 5 × 10⁶) as a dispersion | | |
| in a concentrated saline aqueous solution | | |
| (Ultimer from Ondeo) | | |
| Behenyltrimethylammonium chloride | — | 1.5% AM |
| Cetyltrimethylammonium chloride | 2.5% AM | — |
| Cetylstearyl alcohol | — | 1.5% AM |
| Amodimethicone | 1.5% AM | — |
| (DC 939 from Dow Corning - 35% AM) | | |
| Distarch phosphate | 8% | — |
| Polyquaternium-37/Mineral oil/PPG1 Trideceth 6 | — | 1.5% AM |
| (Salcare SC 95 from Ciba) | | |
| NaCl | 2% | — |
| Water qs | 100% | 100% |

1. A cosmetic composition comprising, in a cosmetically acceptable medium, at least one cationic surfactant, at least one non-cellulose-based thickening polymer, and at least one drawing polymer with a drawing power of greater than 5 cm.

2. The composition according to claim 1, wherein the at least one drawing polymer is chosen from:
(a1), a dispersion of particles of at least one water-soluble cationic polymer with a weight-average molecular mass of greater than 10⁶ in a saline aqueous solution, obtained by heterogeneous free-radical polymerization of at least one water-soluble monomer with precipitation of the polymer formed, and
(a2), a saline aqueous solution of at least one water-soluble cationic polymer with a weight-average molecular mass of greater than 10⁶, obtained by heterogeneous free-radical polymerization of at least one water-soluble monomer with precipitation of the polymer formed.

3. The composition according to claim 2, wherein the at least one water-soluble monomer is chosen from cationic and nonionic monomers comprising at least one ethylenic double bond, and mixtures thereof.

4. The composition according to claim 3, wherein the nonionic monomers are chosen from acrylamide, methacrylamide, N-vinylformamide, N-vinylacetonamide, hydroxypropyl acrylate, and hydroxypropyl methacrylate.

5. The composition according to claim 3, wherein the cationic monomers are chosen from di(C₁₋₄ alkyl)diallylammonium salts and the compounds of formula (I) below: 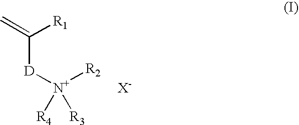
wherein:
R₁ is chosen from hydrogen and methyl;
R₂ and R₃, which may be identical or different, are chosen from hydrogen and linear and branched C₁₋₄ alkyl groups;
R₄ is chosen from hydrogen, linear and branched C₁₋₄ alkyl groups, and aryl groups;
D is chosen from units of the formula: 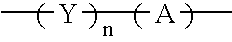
wherein:
Y is chosen from amide, ester, urethane, and urea functions,
A is chosen from linear, branched, and cyclic C₁₋₁₀ alkylene groups, which may be substituted or interrupted with a divalent aromatic or heteroaromatic ring, or which may be interrupted with a heteroatom chosen from O, N, S and P, and which may comprise a ketone, amide, ester, urethane or urea function,
n is 0 or 1; and
X⁻ is chosen from anionic counterions.

6. The composition according to claim 2, wherein the water-soluble cationic polymer is polymerized using at least one cationic monomer of formula (I) below: 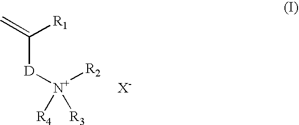
wherein:
R₁ is chosen from hydrogen and methyl;
R₂ and R₃, which may be identical or different, are chosen from hydrogen and linear and branched C₁₋₄ alkyl groups;
R₄ is chosen from hydrogen, linear and branched C₁₋₄ alkyl groups, and aryl groups;
D is chosen from units of the formula: 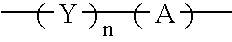
wherein:
Y is chosen from amide, ester, urethane, and urea functions,
A is chosen from linear, branched, and cyclic C₁₋₁₀ alkylene groups, which may be substituted or interrupted with a divalent aromatic or heteroaromatic ring, or which may be interrupted with a heteroatom chosen from O, N, S and P, and which may comprise a ketone, amide, ester, urethane or urea function,
n is 0 or 1; and
X⁻ is chosen from anionic counterions.

7. The composition according to claim 6, wherein the water-soluble cationic polymer is polymerized using a monomer mixture comprising from 0 to 95.5 mole percent of acrylamide and from 4.5 to 100 mole percent of at least one cationic monomer of formula (I).

8. The composition according to claim 6, wherein the water-soluble cationic polymer is polymerized using a monomer mixture consisting of acrylamide and of a cationic monomer of formula (I), the number of moles of the cationic monomer of formula (I) being greater than the number of moles of acrylamide.

9. The composition according to claim 6, wherein the water-soluble cationic polymer is polymerized using a monomer mixture consisting of 10 mole percent of acryloyloxyethyldimethylbenzylammonium chloride and 90 mole percent of acrylamide.

10. The composition according to claim 6, wherein the water-soluble cationic polymer is polymerized using a monomer mixture consisting of 30 mole percent of acryloyloxytrimethylammonium chloride, 50 mole percent of acryloyloxyethyldimethylbenzylammonium chloride, and 20 mole percent of acrylamide.

11. The composition according to claim 6, wherein the water-soluble cationic polymer is polymerized using 10 mole percent of acryloyloxyethyltrimethylammonium chloride and 90 mole percent of acrylamide.

12. The composition according to claim 6, wherein the water-soluble cationic polymer is polymerized using 30 mole percent of diallyldimethylammonium chloride and 70 mole percent of acrylamide.

13. The composition according to claim 2, wherein the concentration of particles of the at least one water-soluble cationic polymer present as a dispersion in a saline aqueous solution in (al) ranges from 0.01% to 20% by weight, relative to the total weight of the dispersion.

14. The composition according to claim 2, wherein the concentration of the at least one water-soluble cationic polymer in a saline aqueous solution in (a2) ranges from 0.01% to 20% by weight, relative to the total weight of the solution.

15. The composition according to claim 2, wherein the saline aqueous solution in (a1) or (a2) comprises at least one anionic salt.

16. The composition according to claim 15, wherein the at least one anionic salt is chosen from ammonium sulfate, ammonium hydrogen sulfate, ammonium chloride, sodium sulfate, sodium hydrogen sulfate, magnesium sulfate, magnesium hydrogen sulfate, aluminium sulfate, and aluminium hydrogen sulfate.

17. The composition according to claim 1, wherein the at least one drawing polymer is present in an amount ranging from 0.01% to 10% by weight, relative to the total weight of the composition.

18. The composition according to claim 1, wherein the at least one cationic surfactant is chosen from optionally polyoxyalkylenated primary, secondary and tertiary fatty amines, quaternary ammonium salts, and mixtures thereof.

19. The composition according to claim 18, wherein the quaternary ammonium salts are chosen from:
those of the general formula (V) below: 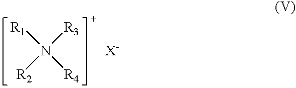
wherein:
R₁, R₂, R₃, and R₄, which may be identical or different, are chosen from linear and branched aliphatic radicals comprising from 1 to 30 carbon atoms, and aromatic radicals; and
X⁻ is chosen from halides, phosphates, acetates, lactates, (C₂-C₆) alkyl sulfates, and alkyl- or alkylaryl-sulfonates;
quaternary ammonium salts of imidazoline;
diquaternary ammonium salts of formula (VII): 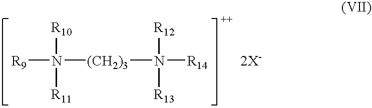
wherein
R₉ is chosen from aliphatic radicals comprising from 16 to 30 carbon atoms;
R₁₀, R₁₁, R₁₂, R₁₃, and R₁₄, which may be identical or different, are chosen from hydrogen and alkyl radicals comprising from 1 to 4 carbon atoms; and
X⁻ is chosen from halides, acetates, phosphates, nitrates, ethyl sulfates, and methyl sulfates;
quaternary ammonium salts comprising at least one ester function.

20. The composition according to claim 19, wherein the quaternary ammonium salts of imidazoline are chosen from those of formula (VI) below: 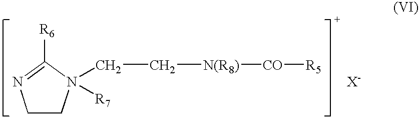
wherein:
R₅ is chosen from alkenyl and alkyl radicals comprising from 8 to 30 carbon atoms;
R₆ is chosen from hydrogen, C₁-C₄ alkyl radicals, and alkenyl and alkyl radicals comprising from 8 to 30 carbon atoms;
R₇ is chosen from C₁-C₄ alkyl radicals;
R₈ is chosen from hydrogen and C₁-C₄ alkyl radicals; and
X⁻ is chosen from halides, phosphates, acetates, lactates, alkyl sulfates, alkyl sulfonates, and alkylaryl sulfonates.

21. The composition according to claim 1, wherein the at least one cationic surfactant is chosen from behenyltrimethylammonium chloride, cetyltrimethylammonium chloride, Quaternium-83, behenylamidopropyl-2,3-dihydroxypropyldimethylammonium chloride, and palmitylamidopropyltrimethylammonium chloride.

22. The composition according to claim 1, wherein the at least one cationic surfactant is present in an amount ranging from 0.05% to 10% by weight, relative to the total weight of the composition.

23. The composition according to claim 1, wherein the at least one non-cellulose-based thickening polymer is of natural or synthetic origin.

24. The composition according to claim 1, wherein the at least one non-cellulose-based thickening polymer is chosen from homopolymers and copolymers comprising ethylenically unsaturated monomers of ester and/or amide type; vinylpyrrolidone homopolymers and copolymers; and non-cellulose-based polysaccharides.

25. The composition according to claim 24, wherein the at least one non-cellulose-based thickening polymer is chosen from acrylic and/or methacrylic acid polymers and copolymers.

26. The composition according to claim 24, wherein the at least one non-cellulose-based thickening polymer is a copolymer of acrylamide or of an acrylamide derivative.

27. The composition according to claim 24, wherein the at least one non-cellulose-based thickening polymer is chosen from crosslinked 2-acrylamido-2-methylpropanesulfonic homopolymers; optionally crosslinked copolymers of acrylamide and of ammonium acrylate; optionally crosslinked homopolymers and copolymers of methacryloyloxyethyltrimethylammonium chloride; and optionally crosslinked, partially or totally neutralized copolymers of acrylamide and of 2-acrylamido-2-methylpropanesulfonic acid.

28. The composition according to claim 24, wherein the at least one non-cellulose-based thickening polymer is chosen from polyethylene glycols and derivatives thereof.

29. The composition according to claim 24, wherein the non-cellulose-based polysaccharides are chosen from glucans, modified or unmodified starches, amylose, amylopectin, glycogen, dextrans, mannans, xylans, arabans, galactans, galacturonans, chitin, chitosans, glucuronoxylans, arabinoxylans, xyloglucans, glucomannans, pectic acids and pectins, arabinogalactans, carrageenans, agars, gum arabics, gum tragacanths, ghatti gums, karaya gums, carob gums, galactomannans, and mixtures thereof.

30. The composition according to claim 1, wherein the at least one non-cellulose-based thickening polymer is present in an amount ranging from 0.001% to 20% by weight, relative to the total weight of the composition.

31. The composition according to claim 1, further comprising at least one additional conditioning agent.

32. The composition according to claim 31, wherein the at least one additional conditioning agent is chosen from silicones, cationic polymers, carboxylic esters comprising at least 12 carbon atoms, plant oils, mineral oils, synthetic oils, and mixtures thereof.

33. The composition according to claim 1, wherein the cosmetically acceptable medium comprises a mixture of water and a cosmetically acceptable solvent.

34. The composition according to claim 33, wherein the cosmetically acceptable solvent is chosen from C₁-C₄ lower alcohols; alkylene glycols; C₅-C₁₀ alkanes; and mixtures thereof.

35. The composition according to claim 1, further comprising at least one additive chosen from anionic, nonionic and amphoteric polymers; thickeners; opacifiers; nacreous agents; vitamins; provitamins; waxes; natural and synthetic ceramides; fragrances; dyes; organic and mineral particles; preserving agents; and pH stabilizers.

36. The composition according to claim 1, wherein the composition is in the form of a conditioner, a composition for permanent-waving, relaxing, dyeing or bleaching the hair, or a rinse-out composition to be applied between the two steps of a permanent-waving or hair-relaxing operation.

37. The composition according to claim 1, wherein the composition is in the form of a rinse-out conditioner.

38. A process for treating keratin materials, comprising applying to the said materials an effective amount of a cosmetic composition comprising, in a cosmetically acceptable medium, at least one cationic surfactant, at least one non-cellulose-based thickening polymer, and at least one polymer with a drawing power of greater than 5 cm, and then optionally rinsing it out.
